(19) United States
(12) Patent Application Publication
(10) Pub. No.: US 2014/0256565 A1
(43) Pub. Date: Sep. 11, 2014

(54) METHODS FOR HLA TYPING

(71) Applicant: LIFE TECHNOLOGIES CORPORATION, Carlsbad, CA (US)

(72) Inventors: DAVID DINAUER, FOX POINT, WI (US); INTA KALVE, MILWAUKEE, WI (US); MARY PARLOW, WAUKESHA, WI (US)

(73) Assignee: LIFE TECHNOLOGIES CORPORATION, CARLSBAD, CA (US)

(21) Appl. No.: 14/185,279

(22) Filed: Feb. 20, 2014

Related U.S. Application Data
(63) Continuation of application No. 11/874,117, filed on Oct. 17, 2007, now abandoned.
(60) Provisional application No. 60/829,867, filed on Oct. 17, 2006.

(51) Int. Cl. C12Q 1/68

(52) U.S. Cl. CPC C12Q 1/6881
USPC 506/2; 435/6.11

(57) ABSTRACT

The present invention relates to methods for reducing the ambiguity in human leukocyte antigen (HLA) allele identification. In particular, the methods comprise using target specific oligonucleotide (TSO) techniques to determine a first set of possible HLA alleles. The methods further comprise using sequence-based typing (SBT) to obtain a second set of possible HLA alleles. The two sets of the possible HLA alleles are then combined to determine at least one common allele identified in the both the TSO and SBT assays, thus reducing the ambiguity associated with current HLA typing procedures.

### CROSS-REFERENCE TO RELATED APPLICATIONS

This application claims priority to U.S. patent application Ser. No. 11/874,117 filed Oct. 17, 2007, and U.S. Provisional Patent Application No. 60/829,867 filed Oct. 17, 2006, which applications are hereby incorporated by reference.

### BACKGROUND OF THE INVENTION

1. Field of the Invention

The present invention relates to methods for reducing ambiguities in identifying human leukocyte antigen (HLA) alleles in a subject.

2. Background of the Invention

A major focus of tissue typing and disease association centers around the human leukocyte antigen (HLA) genes and the alleles encoded by these genes. The HLA antigen complex is divisible into 3 separate regions, identified as the Class I, Class II and Class III HLA genes, and spans approximately 3.5 million base pairs on the short arm of chromosome 6. The HLA genes encompass the most diverse antigenic system in the human genome, encoding literally hundreds of alleles that fall into several distinct subgroups or subfamilies.

For example, within the Class I region exist genes encoding the well-characterized Class I MHC molecules designated HLA-A, HLA-B and HLA-C. In addition, there are nonclassical Class I genes including, but not limited to, HLA-E, HLA-F, HLA-G, HLA-H, HLA-J and HLA-X. HLA-A and HLA-C comprise eight exons and seven introns, whereas HLA-B comprises seven exons and six introns. The DNA sequences of the HLA-A, -B and -C are, generally speaking, highly conserved. Allelic variation, however, occurs predominantly in exons 2 and 3, which encode the functional domains of the molecules and are flanked by introns 1, 2 and 3. The Class II molecules are encoded in the HLA-D region, which comprises several Class II genes and comprises three main subregions: HLA-DR, -DO, and -DP.

HLA typing, therefore, involves sequencing of highly polymorphic regions of genes that inevitably result in ambiguous combinations. It is impossible to discern which allele belongs to which gene unless allele-specific sequencing is involved. Sequence-specific oligonucleotide (SSO) is one method employed in identifying HLA allele types. SSO assays, however, are not always reliable for identifying a single HLA allele. Recently, researchers have also begun using sequence based typing (SBT) to identify the loci and alleles of both Class I and Class II HLA genes. Unfortunately, like the SSO methods, the SBT methods currently available in the art do not allow complete resolution of all HLA alleles at a particular locus, such as HLA-B, because HLA alleles both within and between HLA loci are often closely related. Furthermore, the SBT techniques used for allele identification can be time consuming in that SBT techniques require different reaction conditions and often fail to provide adequate negative and positive controls at initial steps.

The National Marrow Donor Program (NMDP) requirements for ambiguity resolution as well as the historical trend for desired unambiguous allelic typing for all loci indicates a long-felt need for a quick, reliable process to resolve allele ambiguities. Furthermore, because genotype frequency and resolution requirements differ among testing centers, the ideal ambiguity resolution strategy would be capable of being universally applied. There are currently many techniques used for resolving ambiguities, but all such techniques require additional amplification reactions, increasing the cost of the primary typing techniques.

Thus there is a need in the art for methods of efficiently reducing ambiguity in allele ambiguity. The methods of reducing allele ambiguity should, ideally, be cost effective, relatively rapid and capable being universally employed, regardless of the testing center.

### SUMMARY OF THE INVENTION

The present invention relates to methods for reducing the ambiguity in human leukocyte antigen (HLA) allele identification. In particular, the methods comprise using target specific oligonucleotide (TSO) techniques to determine a first set of possible HLA alleles. The methods further comprise using sequence-based typing (SBT) on the amplicon that is generated during the TSO assay to obtain a second set of possible HLA alleles. The two sets of the possible HLA alleles are then combined to determine at least one pair of HLA alleles identified in the both the TSO and SBT assays, thus reducing the ambiguity associated with current HLA typing procedures.

The present invention also relates to computer-implemented methods of comparing HLA typing results from TSO assays and SBT assays to reduce the ambiguity of the identity HLA alleles in a subject.

### BRIEF DESCRIPTION OF THE DRAWINGS

FIG. 1 depicts a diagram of combining ambiguities from a TSO assay and an SBT assay to determine possible common identified alleles.

FIG. 2 depicts a sequencing chromatograph using group specific sequence primers (GSSP).

### DETAILED DESCRIPTION OF THE INVENTION

The present invention relates to methods of reducing ambiguity of the identity of a single or a pair of HLA alleles in a subject. The methods may also be used to determine the identity of other highly polymorphic genes, such as, but not limited to animal MHC complexes. In particular the methods may be used to identify MHC allele identities in non-human primates, murine (both rats and mice), porcine, bovine, equine, feline and canine animals, just to name a few. Other highly polymorphic allelic systems include, but are not limited to, cytochrome P450 systems (CYP), glutathione S-transferase proteins (GSTs), Killer Immunoglobulin-like Receptor (“KIR”) systems and the mycA and mycB systems.

In particular, the methods are useful for identifying the genotype of each HLA allele in a diploid organism. The methods comprise subjecting a sample of a nucleic acid, such as DNA or RNA, to an amplification reaction to create at least one amplicon. The generated amplicon is analyzed using the target-specific oligonucleotide (TSO) to identify a subset of possible HLA alleles. The same amplicon is then sequenced to determine the polynucleotide sequence of the amplicon, which identifies a second set of possible HLA alleles. The TSO subset of possible alleles and the sequencing subset(s) of possible HLA alleles are compared to each other to determine one pair of HLA alleles in the subject.

The methods of the present invention comprise analyzing a nucleic acid, such as DNA or RNA sample, taken from a subject. As used herein, a sample can be any environment that may be suspected of containing the nucleic acid. Thus, a sample includes, but is not limited to, a solution, a cell, a body fluid, a tissue or portion thereof, and an organ or portion thereof. Examples of animal cells include, but are not limited to, insect, avian, and mammalian such as, for example, bovine, equine, porcine, canine, feline, human and nonhuman primates. The scope of the invention should not be limited by the sample type or cell type assayed. Examples of biological fluids to be assayed include, but are not limited to, blood, plasma, serum, urine, saliva, milk, seminal plasma, synovial fluid, interstitial fluid, cerebrospinal fluid, lymphatic fluids, bile and amniotic fluid. The scope of the methods of the present invention may not be limited by the type of body fluid assayed. Indeed, the sample from which the nucleic acid to be amplified derives can encompass blood, bone marrow, spot cards, RNA stabilization tubes, forensic samples, or any other biological sample in which HLA alleles can be amplified. In one embodiment, the sample is a blood sample. In another embodiment, the sample is hair or a hair follicle, a buccal swab, buffy coat or amniocyte. The terms “subject” and “patient” are used interchangeably herein and are used to mean an animal, particularly a mammal, more particularly a human or nonhuman primate. Furthermore, samples taken from individuals can be pooled and analyzed simultaneously. For example, individual samples can be tagged for unique identification and multiple tagged samples can be pooled and the methods of the invention can be performed on the multiple samples simultaneously.

The samples may or may not have been removed from their native environment. Thus, the portion of sample assayed need not be separated or removed from the rest of the sample or from a subject that may contain the sample. Of course, the sample may also be removed from its native environment. For example, the sample may be a tissue section that can be used manipulated to extract any nucleic acid residing therein. Indeed, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes, but is not limited to, freezing and/or preserving the sample prior to assaying.

The nucleic acid to be analyzed can be isolated from the sample using any technique known in the art. In some embodiments, the sample will comprise genomic DNA. In other embodiments, the sample will comprise RNA. In still other embodiments, sample will comprise cDNA. In many embodiments, the nucleic acid will not be isolated from the sample before the amplification reaction. In other embodiments, the nucleic acid will be isolated from the sample prior to the amplification reaction.

The nucleic acid is subject to an amplification reaction. Amplification using primers may be carried out using a variety of amplification techniques, many of which are well-known. Suitable amplification techniques include, but are not limited, to those techniques that use linear or exponential amplification reactions. Such techniques include, but are not limited to, polymerase chain reaction (PCR)-based, transcription based amplification, strand displacement amplification (SDA)-based, rolling circle amplification (RCA)-based and multiple displacement amplification (MDA)-based amplifications. For example, the sample may be subjected to reverse transcriptase PCR (RT-PCR) of HLA mRNA for expression analysis. During amplification, the type of nucleic acid (e.g., RNA, DNA and/or cDNA) amplified by the primers and primers sets is not particularly limiting as long as the primers can hybridize and amplify the target nucleic acid in the sample. One of skill in the art will understand that if cDNA is amplified during an amplification reaction, cDNA will be sequenced during the subsequent sequencing reaction. In some embodiments, RT-PCR will be used to reverse transcribe RNA and amplify the eDNA that results. This RT-PCR method is well-known in the art and several commercial kits exist. In one embodiment, RNA is the nucleic acid that is analyzed.

Any primer or set of primers can be used to generate the amplicon(s). Examples of primers include, but are not limited to Dynal RELI™ SSO Sequencing primers, which are available from Invitrogen Corp. (Carlsbad, Calif., USA). Additional primers are described in United States Pre-Grant Publication No. 2006/0078930, which is incorporated by reference.

As one of skill in the art would recognize, multiplex amplifications may offer significant advantages over non-multiplex amplifications in terms of time and efficiency. Recognizing this, another aspect of the invention provides methods for multiplex amplification of human leukocyte antigen (HLA) alleles based on the use of primer pairs or primer sets capable of simultaneously amplifying multiple alleles from one or more HLA loci. In embodiments encompassing multiplex amplification, primer pairs and sets may be selected to amplify any HLA alleles present in a genomic sample using a multiplex amplification approach. The selection of an appropriate primer pair or primer set for a particular multiplex amplification will depend on the alleles and loci that are to be amplified. An appropriate primer pair or primer set should be selected such that it is capable of amplifying multiple alleles from the selected locus or loci under the same (or very similar) amplification conditions and protocols. Many different combinations of primers may be suitable for use in multiplex applications. In some embodiments, the primers used in multiplex reactions will have 5′ portions with non-homologous sequence.

In some embodiments of the present invention, a multiplex amplification is used to amplify a plurality of portions of a single HLA locus. Generally, where a plurality of portions of a single HLA allele are to be amplified, the primer pairs or sets desirably include a multiplicity of primers that hybridize to multiple non-allele specific regions of the HLA loci. This hybridization to non-allele specific regions allows all different HLA alleles to be successfully amplified. In many cases, following multiplex amplification using the multiplicity of primers, the plurality of amplicons produced will cover some overlapping sequence.

In other embodiments of the present invention, multiplex amplification is used to amplify multiple HLA alleles from two or more HLA loci. This includes embodiments where a multiplex amplification is used to amplify all HLA alleles of two or more HLA loci. Although each HLA locus is physically distinct, with some being separated by large distances, some embodiments provide for all loci to be amplified in a single multiplex reaction which amplifies all or a selected subgroup of clinically significant loci. For example, in some illustrative embodiments all alleles of the two or more HLA loci may be amplified simultaneously in a single vessel by using an appropriate primer set, as provided herein. Where alleles from more than one loci are to be amplified, the primer set desirably includes a primer pair that is specific to each locus to be amplified. In some embodiments, the multiplex amplification of alleles from different HLA loci is achieved while maintaining individual locus specificity because the product sizes produced from the amplification of individual loci differ in size and, therefore, may be separated by, for example, electrophoresis or chromatography.

Different amplification strategies may be employed for amplifying the alleles of different HLA loci. For example, a non-multiplex amplification approach may be sufficient for the amplification of alleles that are relatively easily resolved. Thus, where alleles of the HLA-A locus are being amplified, a non-multiplex amplification may be employed where primers are selected to provide a single amplicon that includes exons 2, 3 and 4. In still other embodiments, the present methods may be used to amplify multiple, and, in some cases, all, alleles of a particular class of HLA loci. For example, the present methods may be employed to amplify multiple alleles of the Class I HLA loci. Similarly, the present methods may be employed to amplify multiple alleles of the Class II HLA loci.

On the other hand, a multiplex amplification may be more desirable when the alleles of a given locus are difficult to resolve. Such may be the case for HLA alleles of the HLA-B locus and HLA alleles for the HLA-DR locus. Thus, where HLA-B locus alleles are being amplified, different primer pairs within a primer set can be used simultaneously to produce dual amplicons that cover exons 2, 3 and 4. The use of two primer pairs in a single amplification of the B locus has the advantage of reducing the number of potential heterozygotic combinations. This results in simplified sequence analysis and a further reduction of the number of resultant ambiguities. These advantages can be achieved, for example, by simultaneously amplifying as two or more distinct groups the regions from exon 1 to intron 3 and intron 3 to exon 5 as two separate products in one amplification mix, resulting in a much more robust amplification than the non-multiplex amplification of a single product. Additionally, amplifying the HLA-B locus as two separate products is advantageous over a single product amplification as a single product is frequently weak, making it difficult to discern using detection methods such as agarose electrophoresis. This difficulty is particularly prominent when modified nucleotides are required. One of skill in the art will understand that when using a multiplicity of primers in multiplex amplification, certain primers in each primer pair can be common. For example, in a multiplex amplification, two (or more) forward primers may be used with a single reverse primer. There is no requirement that an equal number of individual forward and reverse primers be used in each multiplex amplification.

Multiplex amplification can also be used in the amplification of alleles of the HLA-DR locus. Thus, one embodiment of the invention provides a multiplex amplification of alleles of the HLA-DR locus using a primer set that allows for eleven group specific amplifications that achieve resolution of alleles DRB1, DRB3, DRB4, and DRB5 within exon 2. Although the multiplex amplification may possibly consist of amplification of only a single product, plus the HLA control, these reactions can be amplified simultaneously as they require similar or identical reaction conditions. Although resolving exon 2 for DR tissue matching currently has special significance as the standard convention in the transplant community, methods also encompass resolving regions outside of DR locus exon 2.

Another aspect of the invention provides for the use of control primer pairs in HLA allele amplifications. These control primer pairs may be included in the amplifications (non-multiplex and multiplex) to verify the success and accuracy of the amplification. The amplicon produced by amplification using control primer pairs may also be used to specifically identify certain alleles, i.e., the amplicon produced by the control primer pair may be sequenced. Generally, these control primers operate by producing a control amplicon, i.e., a product produced from the amplification of an HLA allele, whenever one or more HLA alleles are present within a sample. Using control primers that amplify an HLA allele is advantageous as they provide a mechanism to ensure that DNA has in fact been added to the amplification reaction. In addition, the control primers may provide an indication of the efficiency of any HLA allele amplification and may identify false positive results. For example, if the results of the amplification provide an amplicon but lack the control amplicon, then the amplicon is likely a false positive. In contrast, if the amplicon and control amplicon are present, then the amplification produced a positive result.

In some embodiments, the control primers amplify a ubiquitous gene in a sample. In these particular embodiments, primers to any gene that can serve as an adequate reaction control may be used. Non-limiting examples include primers that amplify the GAPDH housekeeping genes. In certain embodiments, however, the control primers use target HLA alleles as templates. To provide an effective control, the portion of the HLA allele amplified by the control primer pair is desirably common to all or substantially similar to all HLA alleles being tested. Thus, a control amplicon will be produced if any of the alleles of interest are present. When multiple HLA loci are being amplified with the primer sets of the present invention, a control primer pair common to all or substantially all of the HLA alleles at a particular locus is desirably included for each locus. As long as the control primer pair does not interfere with the primary amplification, the control primer pair can span a region with or without polymorphic positions. Accordingly, the portion of the HLA allele amplified by the control primer pair can have base polymorphisms as well as insertions or deletions. As used herein, a portion of an HLA allele is substantially similar when the control primers are capable of binding to the allele and producing an amplicon.

In additional embodiments, particularly when the target HLA locus is HLA-A, HLA-B, or HLA-C the portion of the HLA allele amplified by the control primer pair comprises all of exon 4 and beyond exon 4. In other embodiments, the control primer pair amplifies all of exon 4 and all of exon 5 of the HLA allele. In yet further embodiments, the control primer pair amplifies all of exon 4, exon 5, exon 6, exon 7, and exon 8. In these embodiments, the primer set can be used in an amplification reaction to amplify an HLA allele and also provide a control. Thus, the presence or absence of a control amplicon in an amplification reaction may be used to confirm the presence or absence HLA alleles in a sample.

The molecular weight of the control amplicon may be predetermined, meaning that the expected size of the product from the control reaction will be known prior to the reaction. Knowing the molecular weight of the control amplicon beforehand allows the user to quickly check for the HLA control amplicon using electrophoresis, e.g., gel electrophoresis, in order to determine the success of the amplification reaction. The size of the control amplicon is not particularly limiting and can be any size capable of amplification and detection, including but not limited to, less than 500, 500-600, 600-700, 700-800, 800-900, 900-1000, or more than 1000 or 2000 base pairs in length.

Following the amplification of the HLA alleles in a sample, the possible alleles may be detected. In one embodiment, detection of alleles in a sample may be carried out using target specific oligonucleotide (TSO) assay. As used herein, the term target-specific oligoucleotide assays include, but are not limited to, sequence-specific oligonucleotides (SSOs), which, as used herein, are probes with nucleotide sequences that are exact matches to the amplicon. The term “target specific oligonucleotides” also encompasses sequence variations in the probes in relation to the amplicon. The TSO assay may or may not be an array. In one embodiment, the TSO assay is a sequence-specific oligonucleotide (SSO) assay, implying a perfect match between the amplicon and the probe. In another embodiment, the TSP assay comprises the probe on a bead. In another embodiment, the TSO assay is an array. In such assays TSOs and/or TSO sets may be contained within distinct, defined locations on a support. The skilled artisan understands that the TSOs may be amplification and/or sequencing primers. Any suitable support can be used for the present assays, such as glass or plastic, either of which can be treated or untreated to help bind, or prevent adhesion of, the TSO. In some embodiments, the support will be a multi-well plate so that the TSOs need not be bound to the support and can be free in solution. Such arrays can be used for automated or high volume assays for target nucleic acid sequences.

In some embodiments, the TSOs will be attached to the support in a defined location. The TSOs can also be contained within a well of the support. Each defined, distinct area of the array will typically have a plurality of the same TSOs. As used herein the term “well” is used solely for convenience and is not intended to be limiting. For example, a well can include any structure that serves to hold the nucleic acid TSOs in the defined, distinct area on the solid support. Non-limiting examples of wells include, but are not limited to, depressions, grooves, walled surroundings and the like. In some of the arrays, TSOs at different locations can have the same probing regions or consist of the same molecule. This embodiment is useful when testing whether nucleic acids from a variety of sources contain the same target sequences. In many embodiments, the solid support will comprise beads known in the art. The arrays can also have TSOs having one or multiple different TSO regions at different locations within the array. In these arrays, individual TSOs can recognize different alleles with different sequence combinations from the same positions, such as, for example, with different haplotypes. This embodiment can be useful where nucleic acids from a single source are assayed for a variety of target sequences. In certain embodiments, combinations of these array configurations are provided such as where some of the TSOs in the defined locations contain the same TSO regions and other defined locations contain TSOs with TSO regions that are specific for individual targets.

The presence or absence of an amplicon may also be determined by standard separation techniques including electrophoresis, chromatography (including HPLC and denaturing-HPLC), or the like. Primers labeled with a detectable moiety may be used in some detection schemes. Suitable examples of detectable labels include fluorescent molecules, beads, polymeric beads, fluorescent polymeric beads and molecular weight markers. Polymeric beads can be made of any suitable polymer including latex or polystyrene. One of skill in the art understands that any detectable label known in the art may be used with the primers and primer sets as long as the detectable label does not interfere with the primers, primer sets or methods of the invention.

In one embodiment, the amplicon is detected using an array. In array assays the amplicons will generally be denatured to form single-stranded nucleic acids, and one of the amplicon strands will be hybridized to the immobilized TSOs on the array. Therefore detecting the hybridization event of a target stand to an addressed TSO array will determine the identities of the first subset of possible alleles. In some embodiments, the amplicon nucleic acid is labeled with a detectable label. In more specific embodiments, the detectable label is inserted into the amplicon during amplification. In even more specific embodiments, the primers used to generate the amplicon are labeled.

As used herein a label is intended to mean a chemical compound or ion that possesses or comes to possess or is capable of generating a detectable signal. Examples of labels includes, but are not limited to, radiolabels, such as, for example, ³H and ³²P, that can be measured with radiation-counting devices; pigments, dyes or other chromogens that can be visually observed or measured with a spectrophotometer and fluorescent labels (fluorophores), where the output signal is generated by the excitation of a suitable molecular adduct and that can be visualized by excitation with light that is absorbed by the dye or can be measured with standard fluorometers or imaging systems. Additional examples of labels include, but are not limited to, a phosphorescent dye, a tandem dye and a particle. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label also includes a “tag” or hapten that can bind selectively to a conjugated molecule such that the conjugated molecule, when added subsequently along with a substrate, is used to generate a detectable signal. For example, one can use biotin as a label and subsequently use an avidin or streptavidin conjugate of horseradish peroxidate (HRP) to bind to the biotin label, and then use a colorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their colorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9th edition, CD-ROM, (September 2002), which is herein incorporated by reference.

In specific embodiments, the fluorophores of the invention include xanthene (rhodol, rhodamine, fluorescein and derivatives thereof) coumarin, cyanine, pyrene, oxazine and borapolyazaindacene. More specific embodiments include sulfonated xanthenes, fluorinated xanthenes, sulfonated coumarins, fluorinated coumarins and sulfonated cyanines. The choice of the fluorophore attached to the labeling reagent will determine the absorption and fluorescence emission properties of the labeling reagent and immuno-labeled complex. Physical properties of a fluorophore label include spectral characteristics (absorption, emission and stokes shift), fluorescence intensity, lifetime, polarization and photo-bleaching rate all of which can be used to distinguish one fluorophore from another.

In particular embodiments, labeling can occur with arbitrary colorants, which can be coupled to nucleotide triphosphates or to oligonucleotides during or after the synthesis. Examples of colorants include but are not limited to Cy-colorants like Cy3 or Cy5 (Amersham Pharmacia Biotech, Uppsala, Sweden), Alexa colorants like Texas Red, Fluorescein, Rhodamine (Molecular Probes, Eugene, Oreg., USA) or lanthanides like samarium, ytterbium and europium (EG&G Wallac, Freiburg, Germany). In one particular embodiment, the primers are fluorescence-labeled primers. In a more particular embodiment, the label is a Cy-colorant. Examples of dyes useful for sequencing and/or array methods are disclosed in U.S. Pat. Nos. 5,847,162; 5,863,727; 5,945,526; 6,335,440; 6,849,745 and United States Pre-Grant Publication No. 2005/0069912, all of which are incorporated by reference.

In additional embodiments detection of hybridization comprises methods which yield, as a result of an adduct having a certain solubility product, a precipitation product. The precipitation product can, but need not be, a colored product. For example, enzymes catalyzing the conversion of a substrate into a less soluble product can be used in this labeling reaction. Examples for such reactions are the conversion of chromogenic substrates like tetramethylbenzidine (TMB)-containing, precipitating reagents by peroxidases, e.g., horseradish peroxidase (HRP) and the conversion of BCIP/NBT mixtures by phosphatases, respectively. Examples for highly sensitive detection methods, wherein a less soluble product is formed, are the Tyramide Signal Amplification System (TSA) and the Enzyme-Labeled Fluorescence (ELF) signal amplification described in The Molecular Probes, Handbook of Fluorescent Probes and Research Products, 9. Auflage, 152-166.

Hybridization detection on an addressed array should reveal the first subset of possible HLA alleles in the nucleic acid sample. By knowing the identity of the sequence/allele at each location on the array, a positive signal at a particular location will indicate the possibility of the allele's identity in the sample. In this manner, the TSO assay will generate a subset of possible HLA alleles. As used herein, the phrases “TSO subset” or “TSO subset of alleles” or “TSO subset of possible alleles” are intended to mean a subset of possible alleles in the subject where the members are identified using TSO techniques. A TSO subset may have more than one set of possible alleles. Of course, the lack of hybridization will also indicate negative data, which is also valuable in that the certain alleles can be eliminated from consideration.

To reduce ambiguity in the identity of the alleles within the TSO subset of alleles, the amplicon is then sequenced in a sequence-based typing (SBT) assay to generate a polynucleotide sequence of at least a portion of the amplicon. Alternatively, the SBT assay may be performed on an amplicon or portion of an amplicon that was not used in the TSO assay. Any SBT technique designed to sequence at least a portion of the amplicon will suffice, and the entire amplicon need not be sequenced. Examples of SBT techniques include but are not limited to chemical sequencing, chain termination methods, dye termination methods, pyrosequencing methods and even nanopore sequencing methods. In one embodiment, the amplicon is sequenced using a dideoxy termination method, which is well known in the art. One example of a sequencing method that may be used for SBT analysis of the amplicon is a dideoxy termination method utilizing energy transfer (ET) exemplified, for example in Amersham's ET methods and kits.

In one embodiment of the present invention, the sequencing of the amplicon comprises the use of Group Specific Sequencing Primers (GSSPs). As used herein, GSSPs are primers are designed to anneal just outside of a highly polymorphic region of an HLA gene sequence. The GSSP primers can anneal to a portion of an exon of the target HLA allele or to a portion of an intron of the HLA allele.

In another embodiment, the particular GSSPs are chosen based upon the members of the TSO subset of alleles. If the GSSPs are chosen based upon the members of the TSO subset of alleles, the determination of which GSSPs to use in the SBT assay may be computer or machine-implemented. In one particular embodiment, the GSSPs that are chosen to be used in the SBT assay are Class I primers. In another particular embodiment, the GSSPs are Class II. In yet another particular embodiment, the GSSPs are Class III.

The SBT assays generate a subset of possible HLA alleles from the polynucleotide sequences generated in the SBT assay. As used herein, the phrases “sequencing subset” or “sequencing subset of alleles” or “sequencing subset of possible alleles” are intended to mean a subset of possible HLA alleles in the subject where the members are identified using SBT techniques. A sequencing subset of alleles may have more than one set of possible alleles.

Once the TSO subset of alleles and at least one sequencing subset of alleles have been identified, the subsets are combined or compared with one another to determine alleles that are common to the TSO subset and sequencing subset(s) of alleles. The pair of alleles that is or would be common to all subsets of identified alleles will be the identity of each HLA allele in the subject. In one embodiment, a comparison of one TSO subset of alleles and one sequencing subset of alleles yields only one pair of possible alleles that is common to both subsets. In this embodiment, therefore, only one SBT assay needs to be performed.

In another embodiment, a comparison of one TSO subset of alleles and one sequencing subset of alleles yields more than one pair of possible alleles that is common to both subsets. In such a case where a comparison of one TSO subset and one sequencing subset yields more than one pair of possible HLA alleles, a subsequent SBT assay may be performed on different portions of the amplicon. Indeed, the same amplicon generated in the TSO assay can be used in all SBT assays. If a subsequent SBT assay is performed, the additional SBT assays will generate one or more additional polynucleotide sequences of the amplicon that can be used to generate subsequent sequencing subsets of possible alleles. Depending upon the results of the first comparison between the TSO subset and SBT subset, it may or may not be necessary to generate more than one subsequent polynucleotide sequence to positively identify the pair of alleles in the subject. In one embodiment, only one subsequent polynucleotide sequence is generated in any subsequent SBT assay to positively identify the HLA alleles in the subject. In another embodiment, more than one polynucleotide sequence is generated in subsequent SBT assays to positively identify the HLA alleles in the subject. The invention therefore encompasses embodiments where any number of subsequent SBT assays may be performed such that the methods may generate and utilize one, two, three, four, five, six, seven, eight or more sequencing subsets of alleles.

The comparison of the subsets of alleles may be performed in any manner that allows the identification of common alleles identified by all assays. Thus, the comparison may be done with or without the aid algorithms, e.g., a computer program, that can identify members of alleles common to all subsets. In addition, software methods may be used to interpret the TSO data and the SBT data simultaneously. Examples of software that may aid in the analysis are disclosed in Helmberg, W., et al., Tissue Antigens 51(6):587-92 (June, 1998), which is incorporated by reference.

Thus, the present disclosure makes it clear to those of skill in the art that determining allele identity may be assisted or performed by software configured to analyze the various subsets of data. The invention therefore also provides for programming software that is capable of analyzing and comparing the various subsets of possible alleles to positively identify the pair of HLA alleles in the subject.

In one particular embodiment, the computer readable media comprises instructions for (a) storing into memory results obtained from a target-specific oligonucleotide (TSO) assay performed on the nucleic acid sample; (b) storing into memory results obtained from at least one sequence-based typing (SBT) assay performed on the amplicon that is generated during the TSO assay and (c) determining the allele identity, wherein the instructions for determining the allele identity comprise combining the TSO results and the SBT results to determine the allele identities. The computer readable medium may further comprise instructions for displaying the results of the subset comparison to a user through, for example, a graphical user interface such as a computer screen.

Portions of such software may include commercially-available software tools. For instance, a software package such as uTYPE™ HLA sequencing software, available from Invitrogen Corp. To perform the data analysis and data comparison, one may perform the calculations and methodology as described herein by using any number of programming languages. For example, a stand-alone program written in, for instance, C++, visual basic, FORTRAN, or the like may be used to combine and analyze the TSO data set and the SBT data set(s). Alternatively, a specialized script, configured to perform data subset comparison, may be written for use with another commercially-available data analysis software package. To display the allele identities, any software tool or program may be used, as will be apparent to those having skill in the art.

This software may be integrated into a computer, into a genetic sequencing system, into a microarray reader, into a hand-held device, or the like, depending on the application. For instance, in a clinical setting, the software may be hosted on a lab computer workstation or computer server. In another clinical setting, the software may be resident on a client computer or on a hand-held device. Alternatively, the software may run within the sequencing system itself so that a single unit could be used for the gather the sequencing subset of alleles and to display the final allele identities. The software may also analyze the TSO subset and display particular sequencing primers that may be used in the SBT assay. The software may also combine the TSO subsets and the initial SBT subsets to display particular sequencing primers that can be used in any subsequent SBT assay to generate subsequent or additional sequencing subsets of alleles.

The following examples are intended to illustrate select embodiments of the present invention and should not be construed to limit the scope of the invention in any way.

### EXAMPLES

### Example 1

### Target-Specific Oligonucleotide (TSO) Assay to Identify Possible HLA Alleles

Genomic DNA was collected from a sample subject and the Class I HLA alleles present in the genome were analyzed using the RELI™ HLA typing kit available from Invitrogen Corporation (Carlsbad, Calif., USA) according the manufacturers suggested protocol. Briefly, genomic DNA was isolated and purified to an optical density of at least about 1.7 OD and was subsequently digested with a restriction enzyme. The fragments were then subjected to PCR amplification using biotinylated amplification primers contained in the RELI™ kits.

The amplicons were purified and analyzed using an TSO assay, in particular using sequence-specific oligonucleotides (SSOs). After annealing onto the test strips, unhybridized nucleic acids were washed and color was developed using a streptavidin-horseradish peroxidase conjugate solution.

The pattern of color development was then analyzed. Based upon the analysis, the SSO subset of possible alleles was determined to be A*0101+A*24; A*0102+A*2402; A*0106+A*24; A*0110+A*24; A*0112+A*24; A*0112+A*2423; A*0112+A*2446; A*010101/0104N+A*240301/2433 and A*2446+A*3601.

### Example 2

### Sequence-Based Typing (SBT) Assay on the Amplicon from Example 1

The amplicons from Example 1 were sequenced using the SECORE™ GSSP sequencing kit, available from Invitrogen Corporation. Briefly, the remaining amplicons not used in the SSO reaction were purified and subjected to dideoxy chain termination sequencing using the sequencing primer contained within the SECORE™ GSSP kit. The primers in the SECORE™ GSSP kits are group specific sequencing primers meaning that the primers are selected to sequence a highly polymorphic area of the HLA alleles, i.e., a group of HLA alleles, such that the maximum number of possible alleles across samples may be amplified with a minimum of sequencing primers.

The sequence was generated using uTYPE™ available from Invitrogen Corporation. Based upon the sequence information generated from the SBT assay, the uTYPE™ software generated the sequencing subset of possible alleles, which was determined to be A*0114+A*2410; A*24G1+A*3604; A*010101/0104N+A*240301/2433 and A*2446+A*3601.

### Example 3

### Comparison of Results from TSO and SBT Assays to Determine Commonly Identified Alleles

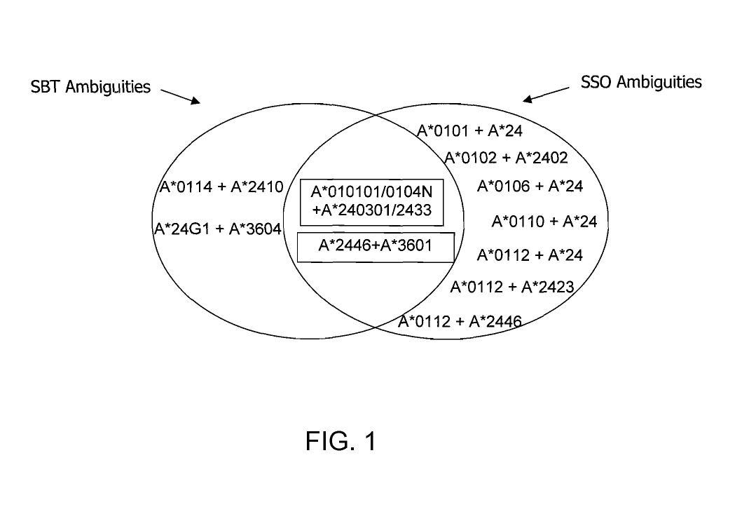

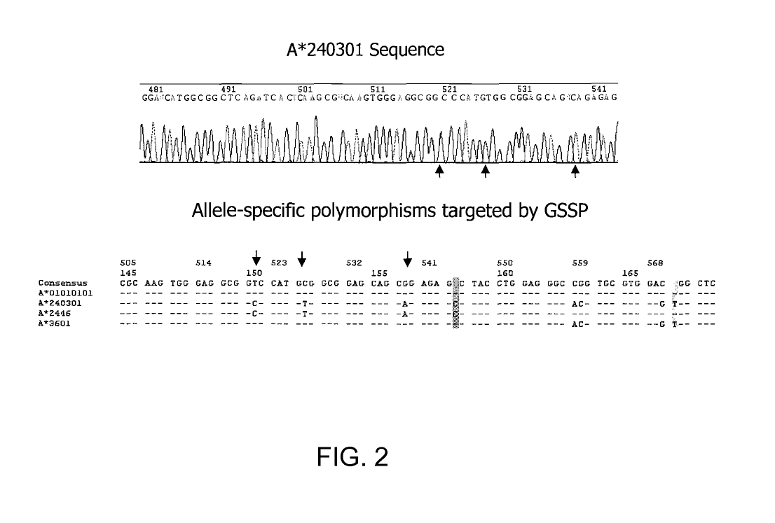

Referring to FIGS. 1 and 2, FIG. 1 demonstrates the comparison or combination of a single subset of SSO alleles (right circle) and a single subset of sequencing alleles (left circle). The comparison identified two pairs of alleles common to both subsets, of which the first pair was identified as A*010101/0104N+A*240301/2433 (top box within the circles), and the second pair was identified as A*2466+A*3601 (lower box within the circles). Thus, the combination of the TSO results with one set of SBT results drastically narrowed the possible pair of alleles down to two.

The amplicon was then subjected to a subsequent SBT assay after the initial SBT assay. In the subsequent SBT assay, the GSSPs that targeted the A*24 allele group were utilized to generate an allele identity of A*240301 (FIG. 2). At this point, the investigator was able to eliminate the second possibility (lower box in FIG. 1) because second pair of possible alleles did not contain the A*240301 allele. Accordingly, the identity of the HLA alleles was positively identified as A*010101/0104N+A*240301/2433 because the A*240301 allele was common to all subsets of possible alleles.

What is claimed is:

1. A method of positively identifying HLA alleles in a subject, the method comprising
a) subjecting a nucleic acid sample to an amplification reaction to create at least one amplicon, said nucleic acid sample being taken from said subject;
b) denaturing and hybridizing the at least one amplicon to one or more target-specific oligonucleotide (TSO);
c) detecting the hybridization of a strand of the amplicon to the TSO;
d) identifying a TSO subset of possible alleles, wherein the TSO subset of possible alleles is based upon the detected hybridization of the amplicon to a specific TSO;
e) subjecting the amplicon to at least one sequencing assay to determine the polynucleotide sequence of the amplicon;
f) identifying at least one sequencing subset of possible alleles, wherein the at least one sequencing subset of possible alleles is based upon the polynucleotide sequence of the amplicon; and
g) making at least one comparison of the TSO subset of possible alleles with the at least one sequencing subset of possible alleles to determine alleles identified in all subsets of alleles,
wherein the positive identity of the HLA alleles in the subject are the alleles common to all subsets of possible alleles.

2. The method of claim 1, wherein the nucleic acid sample comprises DNA.

3. The method of claim 1, wherein the nucleic acid sample comprises RNA.

4. The method of claim 1, wherein the amplification reaction is selected from the group consisting of polymerase chain reaction (PCR)-based, strand displacement amplification (SDA)-based, rolling circle amplification (RCA)-based and multiple displacement amplification (MDA)-based.

5. The method of claim 4, wherein said amplification reaction is a multiplex reaction.

6. The method of claim 4, wherein said amplification reaction is a not a multiplex reaction.

7. The method of claim 4, wherein the amplification reaction is PCR-based.

8. The method of claim 7, wherein the amplicon comprises at least one detectable label.

9. The method of claim 8, wherein the label is selected from the group consisting of biotin, streptavidin, avidin, alkaline phosphatase, horseradish peroxidase and a fluorophore.

10. The method of claim 4, wherein hybridization to the TSO occurs on an array or on a bead.

11. The method of claim 4, wherein the sequencing assay comprises the use of group specific sequence primers (GSSPs).

12. The method of claim 11, wherein the GSSPs are Class I GSSP primers.

13. The method of claim 4, wherein a comparison of the TSO subset and one sequencing subset identifies only one pair of HLA alleles in the subject.

14. The method of claim 4, wherein a comparison of the TSO subset and one sequencing subset identifies more than one possible pairs of HLA alleles in the subject.

15. The method of claim 14, wherein the amplicon is subjected to more than one sequencing assay to generate more than one sequencing subsets of possible alleles.

16. The method of claim 15, wherein the more than one sequencing reactions comprise the use of group specific sequence primers (GSSPs).

17. The method of claim 16, wherein the GSSPs are Class I GSSPs.

18. The method of claim 17, wherein a comparison of the TSO subset and the more than one sequencing subsets of possible alleles identifies only one pair of HLA alleles in the subject.

19. The method of claim 4, wherein the comparison occurs in a processor comprising machine executable instructions configured to combine the TSO results and the SBT results.

20. A method for reducing the ambiguity of the identity of an HLA allele in a subject, the method comprising
a) obtaining results from a target-specific oligonucleotide (TSO) assay performed on a nucleic acid sample taken from the subject;
b) obtaining results from at least one sequence-based typing (SBT) assay performed on the amplicon derived from the TSO reaction; and
c) combining said TSO results and the at least one set of SBT results,
wherein the combination identifies one pair of HLA alleles in the subject.

21. The method of claim 20, wherein more than one set of SBT results are combined to determine the allele identity.

22. The method of claim 20, wherein the combination occurs in a processor with machine executable instructions configured to combine the TSO results and the SBT results.

23. A computer readable medium containing program instructions for determining allele identity of a nucleic acid sample, the computer readable media comprising
a) instructions for storing into memory results obtained from a target-specific oligonucleotide (TSO) assay performed on the nucleic acid sample;
b) instructions for storing into memory results obtained from at least one sequence-based typing (SBT) assay performed on the amplicon that is generated during the TSO assay; and
c) instructions for determining the allele identity, wherein the instructions for determining the allele identity comprise combining the TSO results and the SBT results to determine the allele identities.

24. The computer readable medium of claim 23, wherein the instructions for determining the allele identity, further comprise combining more than one set of SBT results with the TSO results.


SEQUENCE LISTING (2 sequences)

SEQ ID NO: 1 - DNA, 68 bp, Artificial Sequence
  A*240301 Sequence
ggatcatggcggctcagatcactcaagcgcaagtgggaggcggcccatgtggcggagcagtcagagag

SEQ ID NO: 2 - DNA, 64 bp, Artificial Sequence
  Allele-Specific polymorphisms targeted by GSSP
cgcaagtgggaggcggtccatgcgcagcggagagctacctggagggccggtccgtggacggctc